(19) United States
(12) Patent Application Publication
(10) Pub. No.: US 2021/0128159 A1
(43) Pub. Date: May 6, 2021

(54) CLIP APPLIER AND CARTRIDGE

(71) Applicant: TELEFLEX MEDICAL INCORPORATED, Morrisville, NC (US)

(72) Inventors: Warren TAYLOR, Cary, NC (US); Carson J. SHELLENBERGER, Cary, NC (US); Lynn WILLETT, Pittsboro, NC (US); Paul E. WHITING, Wake Forest, NC (US)

(21) Appl. No.: 17/149,958

(22) Filed: Jan. 15, 2021

Related U.S. Application Data
(63) Continuation of application No. PCT/US2019/042390, filed on Jul. 18, 2019.
(60) Provisional application No. 62/700,031, filed on Jul. 18, 2018.

(51) Int. Cl. A61B 17/128; A61B 17/122

(52) U.S. Cl. CPC A61B 17/1285; A61B 17/1222

(57) ABSTRACT

A clip applier may be configured to releasably secure a surgical clip contained in a cavity of a cartridge and having first and second leg members. The clip applier may include first and second jaw members. The first jaw member may include a first clip engagement portion configured to engage the first leg member, and a first cartridge engagement portion including at least one first flange extending laterally of the first clip engagement portion. The second jaw member may include a second clip engagement portion configured to engage the second leg member, and a second cartridge engagement portion including at least one second flange extending laterally of the second engagement portion. The first and second flange(s) may be configured to abut side surfaces of the cartridge to align the first and second jaw members with the first and second leg members.

### PRIORITY

The present application is a continuation of International Patent Application PCT/US2019/042390, filed Jul. 18, 2019, which claims the priority of U.S. Provisional Application 62/700,031, filed on Jul. 18, 2018, the entire disclosures of which are incorporated herein by reference in their entirety.

### TECHNICAL FIELD

The present disclosure relates generally to medical devices, and more particularly to clips appliers and cartridges for applying a surgical clip to tissue.

### BACKGROUND

The ligation of tissue (e.g., blood vessels, lymph nodes, nerves, cystic ducts, or cardiac tissue) is a common practice in many surgical procedures. Current ligation systems often include a surgical clip, a clip applier, and/or a cartridge. The clip applier is used by a user to grasp a surgical clip from a cartridge and to apply the surgical clip to tissue, for example, to ligate a blood vessel (e.g., a vein or an artery).

### OVERVIEW

The present inventors recognize that there is a need to improve one or more features of the clip applier systems. Current clip applier systems do not have completely reliable interfaces between cartridges, surgical clips, and/or jaw members of clip appliers. For example, users often have difficulty in loading the surgical clip onto the clip applier from the cartridge. Alignment of the clip applier with bosses on the surgical clip can be difficult due to the small size of the surgical clip and the lack of alignment features on the cartridge. Insufficient engagement between the two components can cause the surgical clip to be misaligned or fall out during application. If the surgical clip contacts tissue while being positioned inside the body, a proximal portion of the clip may move laterally or “fish-tail” relative to the clip applier because of a lack of stability at the proximal portion of the surgical clip. The disclosed clip appliers, cartridges, and/or clip applier systems are directed to mitigating and/or overcoming one or more of these problems, and/or other problems of the prior art.

A first aspect of the present invention is directed to a clip applier configured to releasably secure a surgical clip contained in a cavity of a cartridge, the surgical clip may include first and second leg members, the clip applier may include: a first jaw member having a first clip engagement portion configured to engage the first leg member, and a first cartridge engagement portion having at least one first flange extending laterally of the first clip engagement portion, the at least one first flange configured to abut a first side surface of the cartridge to align the first jaw member with the first leg member; and a second jaw member having a second clip engagement portion configured to engage the second leg member, and a first cartridge engagement portion having at least one second flange extending laterally of the second engagement portion, the at least one second flange configured to abut a second side surface of the cartridge to align the second jaw member with the second leg member.

In some embodiments of the clip applier, the first cartridge engagement portion may have a width greater than a width of the first clip engagement portion, and the second cartridge engagement portion may have a width greater than a width of the second clip engagement portion. In some embodiments of the clip applier, the first clip engagement portion and the second clip engagement portion may be configured to be inserted into the cavity to engage the surgical clip while the at least one first flange and the at least one second flange remains outside of the cavity. In some embodiments of the clip applier, the first clip engagement portion may include at least one recess configured to receive a boss on the first leg member, and the second clip engagement portion may include at least one recess configured to receive a boss on the second leg member. In some embodiments of the clip applier, the at least one recess of the first clip engagement portion may include first and second spaced apart recesses configured to receive first and second bosses on the first leg member, and the at least one recess of the second clip engagement portion may include first and second spaced apart recesses configured to engage first and second bosses on the second leg member. In some embodiments of the clip applier, the at least one first flange may include a pair of first flanges extending in opposing lateral directions of the first clip engagement portion, and the at least one second flange may include a pair of second flanges extending in opposing lateral directions of the second clip engagement portion. In some embodiments of the clip applier, the at least one first flange may be at least partially tapered, and the at least one second flange may be at least partially tapered. In some embodiments of the clip applier, the clip applier may further include a stabilizing portion of at least one of the first and second jaw members, the stabilizing portion configured to laterally stabilize a proximal portion of the surgical clip. In some embodiments of the clip applier, the stabilizing portion may include first and second protrusions, the clip applier being configured to receive a proximal portion of the surgical clip between the first and second protrusions. In some embodiments of the clip applier, the stabilizing portion may extend from an attachment portion of the at least one of the first and second jaw members, and the stabilizing portion may have a width that is less than a width of the attachment portion. In some embodiments of the clip applier, the at least one first flange and the at least one second flange may form a V-shape when the clip applier is in an open configuration.

A second aspect of the present invention is directed to a method of loading a clip applier with a surgical clip from a cartridge. The surgical clip may include first and second leg members, and the clip applier may include first and second jaw members. The method may include: abutting at least one first flange of the first jaw member on a first side surface of the cartridge to align the first jaw member with the first leg member; abutting at least one second flange of the second jaw member on a second side surface of the cartridge to align the second jaw member with the second leg member; engaging the first leg member with a first engagement portion of the first jaw member; and engaging the second leg member with a second engagement portion of the second jaw member.

In some embodiments of the method, the method may further include receiving a first boss in at least recess of the first jaw member to engage the first leg member, and receiving a second boss in at least one recess of the second jaw member to engage the second leg member. In some embodiments of the method, abutting at least one first flange of the first jaw member may include abutting a pair of first flanges extending laterally of the first engagement portion against the first side surface, and abutting at least one second flange of the second jaw member may include abutting a pair of second flanges extending laterally of the second engagement portion against the second side surface. In some embodiments of the method, the method may further include inserting a stabilizing portion of the clip applier into a recess of the cartridge to laterally stabilize a proximal portion of the surgical clip with the stabilizing portion.

A third aspect of the present invention is directed to a cartridge for a surgical clip. The cartridge may include: a base; and a plurality of walls extending from the base, the plurality of walls being spaced apart by at least one cavity configured to receive the surgical clip, the plurality of walls comprising first and second side surfaces defining an angle between about 15 and 30 degrees, the first and second side surfaces being configured to engage a clip applier in an open configuration during loading of the surgical clip.

In some embodiments of the cartridge of the third aspect, the angle may be between about 18 and 25 degrees. In some embodiments of the cartridge of the third aspect, the side surfaces may be outside of the at least one cavity. In some embodiments of the cartridge of the third aspect, the cartridge may further include a support member configured to support the surgical clip, the support member extending between the plurality of walls. In some embodiments of the cartridge of the third aspect, the cartridge may further include at least one recess configured to receive a stabilizing portion of the clip applier during loading of the surgical clip, the at least one recess extending along at least one of the plurality of walls. In some aspects of the cartridge of the third aspect, the side surfaces may extend from the base. In some embodiments of the cartridge of the third aspect, the cartridge may further include a flexible insert configured to engage the surgical clip when received in the at least one cavity.

A fourth aspect of the present invention is directed to a cartridge for a surgical clip. The cartridge may include: a base; first and second walls extending from the base; a cavity between the first and second walls, the cavity being configured to receive the surgical clip; a support member extending between the first and second walls, the support member being configured to support the surgical clip; and at least one recess configured to receive a jaw member of a clip applier during loading of the surgical clip, the at least one recess extending along at least one of the first and second walls.

In some embodiments of the cartridge of the fourth aspect, the at least one recess may include a first recess between the support member and the first wall, and a second recess between the support member and the second wall. In some embodiments of the cartridge of the fourth aspect, the cartridge may further include at least one recessed portion defining the at least one recess. In some aspects of the cartridge of the fourth aspect, the at least one recessed portion may include a first recessed portion between the support member and the first wall, and a second recessed portion between the support member and the second wall. In some aspects of the cartridge of the fourth aspect, the at least one recessed portion may have a length greater than a length of the support member. In some aspects of the cartridge of the fourth aspect, the plurality of walls may include first and second side surfaces defining an angle between about 15 and 30 degrees, the first and second side surfaces being configured to engage a clip applier in an open configuration during loading of the surgical clip.

### BRIEF DESCRIPTION OF THE DRAWINGS

In order that the invention may be readily understood, aspects of this invention are illustrated by way of examples in the accompanying drawings.

FIG. 1 illustrates a side view of an exemplary embodiment of a clip applier according to the present invention.

FIG. 2 illustrates a side view of an exemplary embodiment of the clip applier of FIG. 1 loaded with a surgical clip according to the present invention.

FIG. 3 illustrates a perspective view of the exemplary clip applier of FIGS. 1-2.

FIG. 4A illustrates a perspective view of a first exemplary embodiment of a cartridge containing a surgical clip according to the present invention.

FIG. 4B illustrates aside view of the first exemplary cartridge of FIG. 4A.

FIG. 4C illustrates a top view of the first exemplary cartridge of FIG. 4A-B.

FIG. 5A illustrates a perspective view of the exemplary clip applier of FIGS. 1-3 being loaded with a surgical clip from the first exemplary cartridge of FIGS. 4A-4C.

FIG. 5B illustrates a side view of the exemplary clip applier of FIGS. 1-2 being loaded with a surgical clip (not visible) from the first exemplary cartridge of FIGS. 4A-4C.

FIG. 6A illustrates a perspective view of a second exemplary embodiment of a cartridge according to the present invention.

FIG. 6B illustrates a top view of the second exemplary cartridge of FIG. 6A.

The same reference numbers are used in the drawings and the following detailed description to refer to the same or similar parts.

### DETAILED DESCRIPTION

The present invention is generally directed to cartridges, clip appliers, and/or clip applier assemblies configured to facilitate loading and/or stability of a surgical clip. In some embodiments, the clip applier may include a cartridge engagement portion and a clip engagement portion, on each of first and second jaw members. The cartridge engagement portion may include one or more flanges extending laterally from a distal portion of jaw members of the clip applier. The one or more flanges may interact with the cartridge during clip loading and/or pick-up. The one or more flanges may form a ‘V’ shape of an acute angle when in an open configuration. Furthermore, in some embodiments, the acute angle may be formed between angled inner surfaces that produce a taper of at least a portion of the one or more flanges. The acute angle of the one or more flanges may match an angle between side surfaces of a wall of the cartridge containing the surgical clip. Therefore, the clip applier may engage and/or mate with a mating shape of the cartridge, and the cartridge may guide the clip appliers to the ideal orientation to consistently and successfully engage the bosses of the clip. The clip engagement portions may also be configured to be inserted into a cavity of the cartridge to securely engage the surgical clip during loading and/or pick-up.

In some embodiments, the clip applier may include a third point of contact between the clip applier and the surgical clip. The third point of contact may be made between one or more protrusions of the clip applier and a proximal portion of the surgical clip to substantially increase the lateral stability of the surgical clip. The one or more protrusions may provide a lateral force to a hinge portion of the surgical clip to eliminate or reduce fish-tailing of the surgical clip during loading and/or application. The cartridge may include one or more recesses configured to receive the protrusions when loading the surgical clip from a cavity of the cartridge. Thus, the cartridge engagement portions may have a lateral width greater than the clip engagement portions and the stabilizing portion to provide a secure mating engagement with the cartridge. The cartridge engagement portions may also have a height in the pivoting direction of the clip applier less than the clip engagement portions and the stabilizing portion such that the clip applier engages the surgical clip in the cavity of the cartridge, while the flanges remain outside of the cavity.

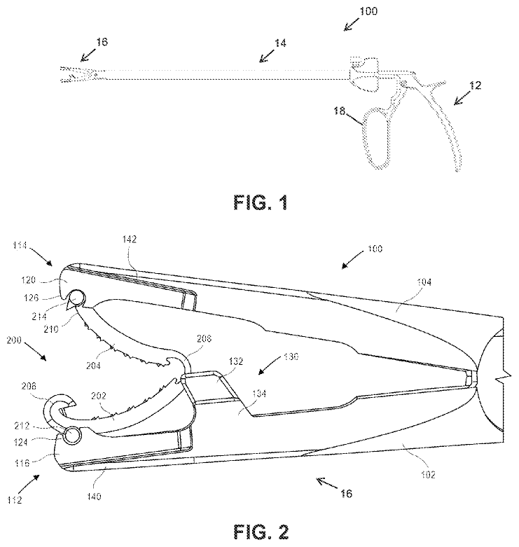

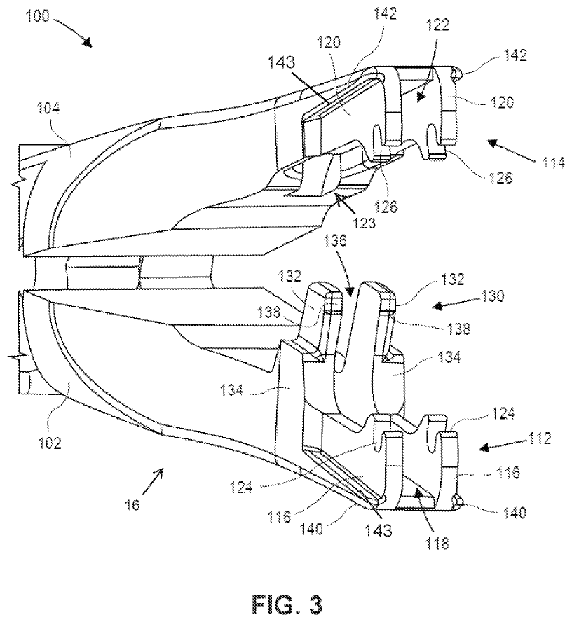

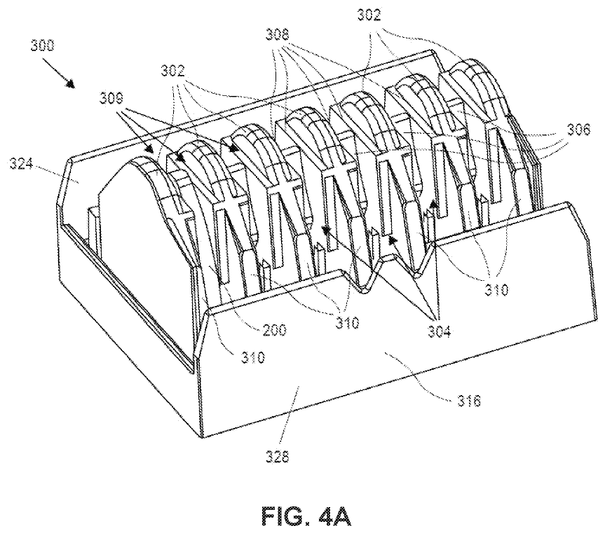

FIG. 1 illustrates a side view of a clip applier 100, FIG. 2 illustrates a side view of the clip applier 100 loaded with a surgical clip 200, and FIG. 3 illustrates a perspective view of the clip applier 100 without the surgical clip 200. As illustrated, the clip applier 100 may include a handle mechanism 12 connected to a proximal end of a shaft 14 and a jaw mechanism 16 pivotally connected to a distal end of the shaft 14. The handle mechanism 12 may include an actuator trigger 18 configured to actuate the jaw mechanism 16 between open and closed configurations.

The surgical clip 200 may include a first leg member 202 and a second leg member 204 pivotally coupled at a hinge portion 206. The surgical clip 200 may include one or more latching or locking mechanisms. For example, the first leg member 202 may include a distal end portion having a hook member 208, and the second leg member 204 may include a distal end portion having a tip member 210. The first leg member 202 and/or the second leg member 204 may be configured to be pivoted relatively to each other between an open configuration and a closed configuration at the hinge portion 206. The hook member 208 may be configured to deflect around the tip member 210 to lock and/or latch the surgical clip 200 in the closed configuration. The surgical clip 200 may further include one or more engagement portions. For example, the first leg member 202 may include one or more bosses 212 on the distal end portion proximal of the hook member 208, and the second leg member 204 may include one or more bosses 214 on the distal end portion at the tip member 210. The one or more bosses 212, 214 may secure to the clip applier 100 during loading and/or application of the surgical clip 200 to tissue. Exemplary embodiments of the surgical clip 200 are further disclosed in U.S. Pat. Nos. 5,100,416 and 9,445,820, the entire disclosures of which are incorporated herein by reference.

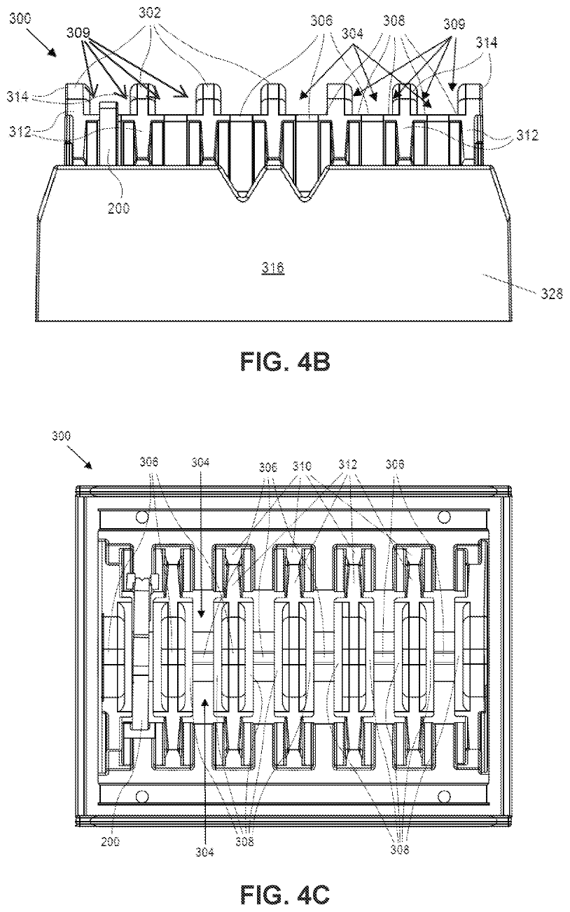

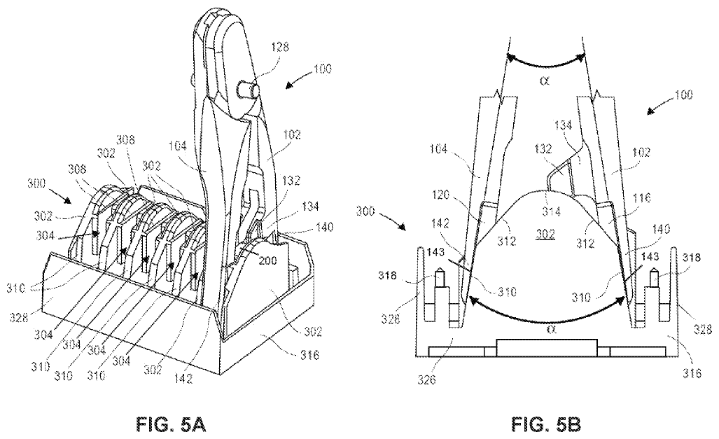

The jaw mechanism 16 of the clip applier 100 may be loaded with the surgical clip 200 from a cartridge 300, 400 (e.g., FIGS. 4A-5B and 6A-B) and apply the surgical clip 200 to tissue. The jaw mechanism 16 may include a first jaw member 102 configured to engage the first leg member 202 and a second jaw member 104 configured to engage the second leg member 204. The first jaw member 102 and/or the second jaw member 104 may be configured to pivot relative to each other, for example, by a hinge pin 228 (e.g., FIG. 5A), in order to move the surgical clip 200 between the open and closed configurations.

The first jaw member 102 may include a first clip engagement portion 112 configured to engage the distal end portion of the first leg member 202 and a second clip engagement portion 114 configured to engage the distal end portion of the second leg member 204. As further illustrated in FIG. 3, the first clip engagement portion 112 may include first and second walls or protrusions 116 spaced apart by a channel 118. The first leg member 202 may be received in the channel 118. Each of the first and second protrusions 116 may have a recess 124 configured to receive the one or more bosses 212 of the first leg member 202. Similarly, the second clip engagement portion 112 may include first and second walls or protrusions 120 spaced apart by a channel 122. The second leg member 204 may be received in the channel 122. Each of the first and second protrusions 120 may have a recess 126 configured to receive the one or more bosses 214 of the second leg member 204.

The first jaw member 102 may include a first cartridge engagement portion including at least one first flange 140 extending laterally of the first clip engagement portion 112. For example, the first jaw member 102 may include a pair of first flanges 140 extending in opposing lateral directions of the first clip engagement portion 112. The second jaw member 104 may include a second cartridge engagement portion including at least one second flange 142 extending laterally of the second clip engagement portion 114. For example, the second jaw member 104 may include a pair of second flanges 142 extending in opposing lateral directions of the second clip engagement portion 112. In other words, the clip engagement portions 112, 114 may extend toward the opposing jaw member 102, 104 in the pivoting direction, and the at least one flange 140, 142 extends transverse of the pivoting direction and peripheral of the clip engagement portions 112, 114. To facilitate discussion of the width of the flange(s) 140, 142, the cartridge engagement portions of each of the jaw members 102, 104 may be considered outer portions including one or more of the opposing flange(s) 140, 142 and the portion of the clip applier 100 attached to and bridging the clip engagement portions 112, 114.

The clip engagement portions 112, 114 and/or the flanges 140, 142 may be configured to align the clip applier 100 with the surgical clip 200 when contained within the cartridge 300, 400, For example, the clip engagement portions 112 may be inserted into a cavity 304, 404 of the cartridge 300, 400 (e.g., FIGS. 4A-5B) and engage an interior surface to prevent and/or reduce lateral and/or rotational movement relative to the cartridge 300, 400. The flanges 140, 142 may engage side surfaces 310 exterior of the cavity 304, 404 of the cartridge 300, 400 (e.g., FIGS. 4A-5B) to guide the clip applier 100 longitudinally relative to the cartridge 300, 400, as further discussed herein. The flanges 140, 142 may have a height in a pivoting direction substantially less than the protrusions 116, 120. The flanges 140, 142 may have an angled inner surface 143 extending along at least a portion of the flanges 140, 142, such that the flanges 140, 142 are at least partially tapered with a gradually reducing height in the distal direction. The angled inner surfaces 143 of the flanges 140, 142 may create a flush engagement with the side surfaces 310 of the cartridge 300, 400, as illustrated in FIG. 5B. In some embodiments, the angled inner surfaces 143 may extend substantially the entire lengths of the flanges 140, 142.

The jaw mechanism 16 may include a stabilizing portion 130 of one or both of the first jaw member 102 and the second jaw member 104. The stabilizing portion 130 may be configured to engage and/or receive a proximal portion of the surgical clip 200 (e.g., the hinge portion 206). For example, the stabilizing portion 130 may include first and second protrusions 132 spaced apart by a channel 136. The proximal portion of the surgical clip 200 may be received within the channel 136, and the first and second protrusions 132 may be configured to apply lateral forces to the proximal portion (e.g., the hinge portion 206) of the surgical clip 200 to laterally stabilize the surgical clip 200 and/or prevent or reduce fish-tailing. The stabilizing portion 130 may not proximally abut the surgical clip 200 to allow the leg members 202, 204 to elongate as the hook member 208 deflects around the tip member 210 during closure. When on the first jaw member 102, the stabilizing portion 130 may be spaced from the second jaw member 104 in an open configuration (FIGS. 1-3) and be received in a widened portion 123 of the channel 122 of the second jaw member 104 in a closed configuration (not shown). However, the stabilizing portion 130 may have other configurations. For example, the first and second protrusions 134 may extend from opposing jaw members 102, 104. In this exemplary embodiment, the first and second protrusions 134 may still be laterally offset to apply lateral forces to the surgical clip 200, while preventing interference when opening and closing the clip applier 100. The channel 136 between the protrusions 132 may be in communication with the channel 118 and/or channel 122. The stabilizing portion 130 may be releasably fixed and/or integral to the at least one of the first jaw member 102 and the second jaw member 104. Exemplary features and embodiments of the stabilizing portion 130 are further disclosed in U.S. patent application Ser. No. 15/927,763, the entire disclosure of which is incorporated herein by reference.

The stabilizing portion 130 may extend from at least one attachment portion 134 of the first jaw member 102. The stabilizing portion 130 may have a width less than a width of the attachment portion 134 to facilitate insertion of the stabilizing portion 130 into the cavity 304, 404 of the cartridge 300, 400 to engage the surgical clip 200. The stabilizing portion 130 may also be configured to align the clip applier 100 with the surgical clip 200 when contained within the cartridge 300, 400. For example, the stabilizing portion 130 may be inserted into the cartridge 300, 400 to receive the surgical clip 200 in the channel 136, while the protrusions 132 is configured to engage an interior surface of the cavity 304, 404 to prevent or reduce lateral and/or rotational movement of the clip applier 100 relative to the cartridge 300, 400 and the attachment portion 134 is maintained outside of the cavity 304, 404. A distal face of the attachment portion 134 may engage a portion of the wall 302.

Thus, to facilitate mating with the cartridge 300, 400, the first cartridge engagement portion may have a width (e.g., width between opposing sides of opposing flanges 140, 142) greater than a width of the first clip engagement portion (e.g., width between opposing sides of opposing protrusions 116, 120) and a width of the stabilizing portion (e.g., width between opposing sides of opposing protrusions 132). The cartridge engagement portions may also have a height in the pivoting direction (e.g., distance between the inner surface 143 and outer surface of the flanges 140, 142) of the clip applier substantially less than a height of the clip engagement portions (e.g., distance between the inner and outer ends of the protrusions 116, 120) and a height of the stabilizing portion (e.g., distance between inner and outer ends of the stabilizing portion 130), such that the clip engagement portions 112, 114 and the stabilizing portion 130 extend further inward into the cavity 304, 404 while the flanges remain outside of the cavity 304, 404. The cartridge engagement portion, the clip engagement portion 112, 114, and/or the stabilizing portion 130 may be integrally formed into each of the first and second jaw members 102, 104.

FIGS. 4A-C illustrate a first embodiment of a cartridge 300 loaded with the surgical clip 200. The cartridge 300 may include a plurality of walls 302 defining one or more cavities 304 therebetween. The one or more cavities 304 may receive one or more surgical clips 200 in a stored configuration. The surgical clip(s) 200 may be supported by one or more support members 306 positioned in the cavities 304. For example, the hinge portion 206 may engage the support member 306, and the first and second leg members 202, 204 may straddle opposing sides of the support member 306. The support members 306 may extend between adjacent walls 302 and/or recessed portions 308.

The walls 302 may include a number of surfaces. For example, the walls 302 may include one or more side surfaces 310, one or more intermediate surfaces 312, and one or more upper convex surfaces 314. The side surfaces 310 may extend from the base 316 to the intermediate surfaces 312, and the intermediate surfaces 312 may be joined at an upper portion by the upper convex surface 314. Each wall 302 may have first and second side surfaces 310 on opposing sides. The first and second side surfaces 310 may be substantially flat and define an acute angle therebetween. The acute angle of the side surfaces 310 may match an acute angle defined by the flanges 140, 142 of the first and second jaw members 102, 104 in an open configuration. Therefore, the first and second surfaces 310 may engage and/or guide the first and second jaw members 102, 104 in an open configuration as the clip applier 100 is inserted into the cartridge 300 to load the surgical clip 200. The angle between the first and second side surfaces 310 may be optimized for the size of the surgical clip 200 and to abut the open configuration of the first and second jaw members 102, 104. In some embodiments, the angle between the first and second side surfaces 310 may be between about 15 and 30 degrees. In some embodiments, the angle between the first and second side surfaces 310 may be between about 18 and 25 degrees. The intermediate surfaces 312 may define a larger angle therebetween than the side surfaces 310 to widen the walls 302 and allow the surgical clip 100 to be received between adjacent walls 302. The side surfaces 310 may be outside or external of the cavity 304 and abut the flanges 140, 142 of the clip applier 100.

One or more notched or recessed portions 308 may extend along the walls 302 and define notches or recesses 309 configured to receive the stabilizing portion 130 of the clip applier 100. The recessed portions 308 may be positioned on opposing ends of the support members 306, between the support members 306 and the walls 302. The recessed portions 308 may have a length extending along the walls 302 greater than that of the support member 306, and the recessed portions 308 may support lateral sides of the leg members 202, 204 of the surgical clip 200. As illustrated, the recessed portions 308 may embody protrusions extending from the walls 302, and the recessed portions 308 may define substantially flat, vertically facing shoulders extending along the walls 302. However, it is contemplated the recessed portions 308 may embody a number of other shapes and configurations. Thus, the recesses 309 may extend vertically between the respective recessed portion 308 and upper convex portion 314, and laterally between the respective support member 306 and wall 302. Although the cavity 314 and the recesses 309 may be described herein as distinct areas, it should be readily apparent that the cavity 314 and recesses 309 may be in communication with each other as a continuous open area of the cartridge 300. Thus, the recesses 309 may be the upper portions of the cavity 314 lateral of the surgical clip 200 and configured to receive or accommodate the stabilizing portion 130. The recesses 309 may produce a width of the cavity 314 extending between the walls 302 at least twice as wide as the surgical clip 200 to receive the stabilizing portion 130 and facilitate the mating of the clip applier 200, as discussed herein.

As further illustrated in FIGS. 4A-C, the base 316 may include a bottom portion 326 and exterior walls 328. The walls 302 may extend from the bottom portion 326 of the base 316 and between the exterior walls 328 of the base 316. The walls 302, support members 306, the recessed portions 308, and the base 316 may be integrally formed. For example, the components may be a unitary plastic body and formed through injection molding. However, it is contemplated that one or more of the components may be formed separately and attached, for example, through welding or an adhesive.

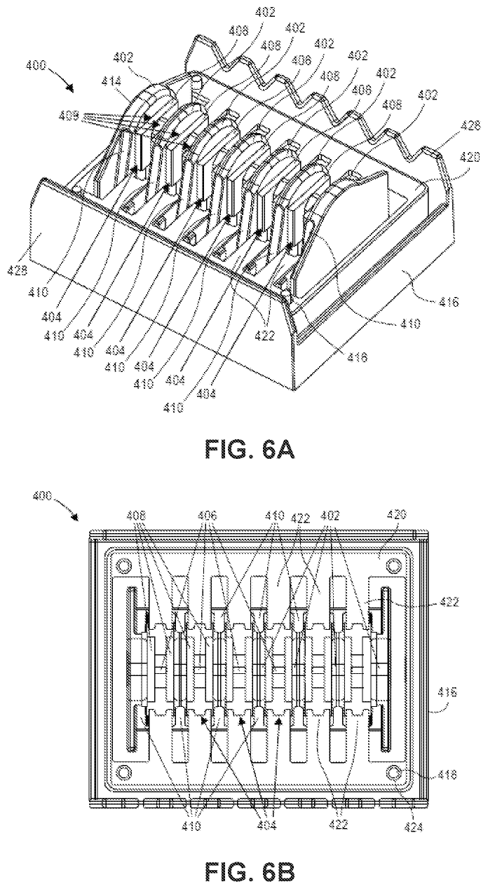

The cartridge 300 may further include a flexible insert having tabs that extend into the cavities 304 and secure the surgical clip 200, as exemplary illustrated with respect to the cartridge 400 in FIGS. 6A-B. The tabs may engage the leg members 202, 204 and/or bosses 212, 214 to prevent the surgical clip 200 from falling out of the cartridge 300. The tabs may be deflected downward as the jaw members 102, 104 are inserted into the cartridge 300 to release the surgical clip 200. The flexible insert may be secured to the base 316 with apertures of the flexible insert that receive pins 318 extending from the base 316.

FIGS. 5A-B illustrate the clip applier 100 being loaded with the surgical clip 200 (not visible) from the cartridge 300. The cartridge 300 may receive the surgical clip 200 in the cavity 304 in a stored configuration. The cartridge 300 may receive the clip applier 100 in an open configuration when the clip applier is loaded with the surgical clip 200. For example, the clip engagement portion(s) 112, 114 of the clip applier 100 may be inserted into the cavity 304 and engage the leg members 202, 204 of the surgical clip 200. The stabilizing portion 130 may be inserted into the recesses 309 and receive the proximal end of the surgical clip 200, while the attachment portions 134 may be sized not to be received into the cavity 304. The flanges 140, 142 may be inserted into the cartridge 300 and abut the side surfaces 310, while remaining outside of the cavity 304. In that sense, the first jaw member 102 and the second jaw member 104 may include a first portion sized to be received into the cavity 304 of the cartridge 300 and a second portion sized to not be received into the cavity 304 of the cartridge 300. Similarly, the stabilizing portion 130 may include a first portion (protrusions 132) sized to be received into the recess 309 of the cartridge 300 and a second portion 134 sized to not be received into the recess 309 of the cartridge 300.

As illustrated in FIG. 5B, an angle α between the flanges 140, 142 when the clip applier 100 is in an open configuration may match the angle α between the side surfaces 310. In some embodiments, the angle α may be between about 15 and 30 degrees. In some embodiments, the angle α may be between about 18 and 25 degrees. The side surfaces 310 may guide the jaw members 102, 104 to align the clip applier 100 and ensure proper engagement of the leg members 202, 204. For example, the side surfaces 310 may ensure that the bosses 212, 214 are properly received in the recesses 124, 126, as illustrated in FIG. 2. The angle α of the flanges 140, 142 may be defined by the angled inner surfaces 143.

FIGS. 6A-B illustrate a second embodiment of a cartridge 400. The cartridge 400 may include features similar to those of the cartridge 300 and similarly indicated in the drawings, that are not discussed herein for sake of brevity. The cartridge 400 may include a plurality of walls 402 defining one or more cavities 404 therebetween. The one or more cavities 404 may receive one or more surgical clips 200 in a stored configuration. The surgical clip(s) 200 may be supported by one or more support members 406 positioned in the cavities 404. For example, the hinge portion 206 may engage the support member 406, and the first and second leg members 202, 204 may straddle opposing sides of the support member 406. The support members 346 may extend between adjacent walls 402 and/or recessed portions 408.

The walls 402 may include a number of surfaces. For example, the walls 302 may include one or more side surfaces 410 and one or more upper convex surfaces 414. The side surfaces 410 may extend from the base 416 and be joined at an upper portion by the convex surface 414. Each wall 402 may have first and second side surfaces 410 on opposing sides. The first and second side surfaces 410 may be substantially flat and define an acute angle therebetween. The acute angle of the side surfaces 410 may match an acute angle defined by the flanges 140, 142 of the first and second jaw members 102, 104 in an open configuration. Therefore, the first and second surfaces 410 may engage and/or guide the first and second jaw members 102, 104 in an open configuration as the clip applier 100 is inserted into the cartridge 400 to load the surgical clip 200. In some embodiments, the angle between the first and second side surfaces 410 may be between about 15 and 30 degrees. In some embodiments, the angle between the first and second side surfaces 410 may be between about 18 and 25 degrees. The walls 402 may include intermediate surfaces (corresponding to intermediate surfaces 312 of FIGS. 4A-5B) having a reduced length, such that the cartridge 400 may be dimensioned to contain smaller surgical clips 200. The side surfaces 410 may be outside or external of the cavity 404 to abut the flanges 140, 142 of the clip applier 100.

One or more recessed portions 408 may extend along the walls 402 and may define recesses 409 configured to receive the stabilizing portion 130 of the clip applier 100. The recessed portions 408 may be positioned on opposing ends of the support members 406, between the support members 406 and the walls 402. The recessed portions 408 may have a length extending along the walls 402 greater than that of the support member 406, and the recessed portions 408 may support lateral sides of the leg members 202, 204 of the surgical clip 200. As illustrated, the recessed portions 408 may embody protrusions extending from the walls 402, and the recessed portions 408 may define substantially flat, vertically facing shoulders extending along the walls 402. However, it is contemplated the recessed portions 408 may embody a number of other shapes and configurations. Thus, the recesses 409 may extend vertically between the respective recessed portion 408 and upper convex portion 414, and laterally between the respective support member 406 and wall 402. Although the cavity 414 and the recesses 409 may be described herein as distinct areas, it should be readily apparent that the cavity 414 and recesses 309 may be in communication with each other as a continuous open area of the cartridge 400. Thus, the recesses 409 may be the upper portions of the cavity 414 lateral of the surgical clip 200 and configured to receive or accommodate the stabilizing portion 130. The recesses 409 may produce a width of the cavity 414 extending between the walls 402 at least twice as wide as the surgical clip 200 to receive the stabilizing portion 130 and facilitate the mating of the clip applier 200, as discussed herein.

As further illustrated in FIGS. 6A-B, the base 416 may include a bottom portion (not shown) and exterior walls 428. The walls 402 may extend from the bottom portion of the base 416 and between the exterior walls 428 of the base 416. The walls 402, the support members 406, the recessed portions 408, and the base 416 may be integrally formed. For example, the components may be a unitary plastic body and be formed through injection molding. However, it is contemplated that one or more of the components may be formed separately and, for example, attached through welding or an adhesive.

The cartridge 400 may include a flexible insert 420 having tabs 422 that extend into the cavities 404 and secure the surgical clip 200 (not shown), as illustrated in FIGS. 6A-B. The tabs 422 may engage the leg members 202, 204 and/or bosses 212, 214 to prevent the surgical clip 200 from falling out of the cartridge 400. The tabs 422 may be deflected downward as the jaw members 102, 104 are inserted into the cartridge 400 to release the surgical clip 200. The flexible insert 420 may be secured to the base 416 with apertures 424 on the flexible insert 420 that receive pins 418 extending from the base 416. Further discussion of the retention of the surgical clip 200 and/or manufacturing of the cartridge 300, 400 can be found in U.S. Pat. No. 6,880,699, the entire disclosure of which is incorporated herein by reference.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A clip applier configured to releasably secure a surgical clip contained in a cavity of a cartridge, the surgical clip comprising first and second leg members, the clip applier comprising:
a first jaw member including: a first clip engagement portion configured to engage the first leg member, and a first cartridge engagement portion including at least one first flange extending laterally of the first clip engagement portion, the at least one first flange configured to abut a first side surface of the cartridge to align the first jaw member with the first leg member; and
a second jaw member including: a second clip engagement portion configured to engage the second leg member, and a second cartridge engagement portion including at least one second flange extending laterally of the second engagement portion, the at least one second flange configured to abut a second side surface of the cartridge to align the second jaw member with the second leg member.

2. The clip applier of claim 1, wherein:
the first cartridge engagement portion has a width greater than a width of the first clip engagement portion, and
the second cartridge engagement portion has a width greater than a width of the second clip engagement portion.

3. The clip applier of claim 1, wherein the first clip engagement portion and the second clip engagement portion are configured to be inserted into the cavity to engage the surgical clip while the at least one first flange and the at least one second flange remain outside of the cavity.

4. The clip applier of claim 1, wherein:
the first clip engagement portion comprises at least one recess configured to receive a boss on the first leg member, and
the second clip engagement portion comprises at least one recess configured to receive a boss on the second leg member.

5. The clip applier of claim 4, wherein:
the at least one recess of the first clip engagement portion comprises first and second spaced apart recesses configured to receive first and second bosses on the first leg member, and
the at least one recess of the second clip engagement portion comprises first and second spaced apart recesses configured to engage first and second bosses on the second leg member.

6. The clip applier of claim 1, wherein:
the at least one first flange comprises a pair of first flanges extending in opposing lateral directions of the first clip engagement portion, and
the at least one second flange comprises a pair of second flanges extending in opposing lateral directions of the second clip engagement portion.

7. The clip applier of claim 1, wherein:
the at least one first flange is at least partially tapered, and
the at least one second flange is at least partially tapered.

8. The clip applier of claim 1, further comprising a stabilizing portion of at least one of the first and second jaw members, the stabilizing portion configured to laterally stabilize a proximal portion of the surgical clip.

9. The clip applier of claim 8, wherein the stabilizing portion comprises first and second protrusions, the clip applier being configured to receive a proximal portion of the surgical clip between the first and second protrusions.

10. The clip applier of claim 8, wherein the stabilizing portion extends from an attachment portion of the at least one of the first and second jaw members, and the stabilizing portion member has a width less than a width of the attachment portion.

11. The clip applier of claim 1, wherein the at least one first flange and the at least one second flange form a V-shape when the clip applier is in an open configuration.

12. A method of loading a clip applier with a surgical clip from a cartridge, the surgical clip comprising first and second leg members, and the clip applier comprising first and second jaw members, the method comprising:
abutting at least one first flange of the first jaw member against a first side surface of the cartridge to align the first jaw member with the first leg member;
abutting at least one second flange of the second jaw member against a second side surface of the cartridge to align the second jaw member with the second leg member;
engaging the first leg member with a first engagement portion of the first jaw member; and
engaging the second leg member with a second engagement portion of the second jaw member.

13. The method of claim 12, further comprising:
receiving a first boss in at least one recess of the first jaw member to engage the first leg member; and
receiving a second boss in at least one recess of the second jaw member to engage the second leg member.

14. The method of claim 12, wherein:
abutting at least one first flange of the first jaw member comprises abutting a pair of first flanges extending laterally of the first engagement portion against the first side surface, and
abutting at least one second flange of the second jaw member comprises abutting a pair of second flanges extending laterally of the second engagement portion against the second side surface.

15. The method of claim 12, further comprising inserting a stabilizing portion of the clip applier into a recess of the cartridge to laterally stabilize a proximal portion of the surgical clip with the stabilizing portion.

16. A cartridge for a surgical clip, the cartridge comprising:
a base; and
a plurality of walls extending from the base, the plurality of walls being spaced apart by at least one cavity configured to receive the surgical clip, the plurality of walls comprising first and second side surfaces defining an angle between about 15 and 30 degrees, and the first and second side surfaces being configured to engage a clip applier in an open configuration during loading of the surgical clip.

17. The cartridge of claim 16, wherein the angle is between about 18 and 25 degrees.

18. The cartridge of claim 16, wherein the first and second side surfaces are outside of the at least one cavity.

19. The cartridge of claim 16, further comprising a support member configured to support the surgical clip, the support member extending between the plurality of walls.

20. The cartridge of claim 16, further comprising at least one recess configured to receive a stabilizing portion of the clip applier during loading of the surgical clip, the at least one recess extending along at least one of the plurality of walls.

21. The cartridge of claim 16, wherein the side surfaces extend from the base.

22. The cartridge of claim 16, further comprising a flexible insert configured to engage the surgical clip when received in the at least one cavity.
